(19) United States
(12) Patent Application Publication
(10) Pub. No.: US 2019/0070210 A1
(43) Pub. Date: Mar. 7, 2019

(54) COMPOSITION AND METHODS FOR CONDITIONS ASSOCIATED WITH CHRONIC PULMONARY OBSTRUCTIVE DISEASE

(71) Applicant: Stephen McNERLIN, Grand Cayman (KY)

(72) Inventor: Stephen McNERLIN, Grand Cayman (KY)

(21) Appl. No.: 15/703,772

(22) Filed: Sep. 13, 2017

Related U.S. Application Data
(63) Continuation-in-part of application No. 15/697,181, filed on Sep. 6, 2017.

(51) Int. Cl. A61K 31/7084

(52) U.S. Cl. CPC A61K 31/7084

(57) ABSTRACT

Embodiments of the present invention disclose a composition comprising nicotinamide adenine dinucleotide (NAD+) and optionally a nonsteroidal anti-inflammatory drug (NSAID), wherein the NAD+is in an amount effective for a condition in a mammal associated with aging. Methods of making and using the invention composition are also disclosed.

### CROSS-REFERENCE TO RELATED APPLICATIONS

This application is a Continuation-in-Part of U.S. Application No. 15/697,181 filed Sep. 6, 2017, the teaching of which is incorporated herein by reference in its entirety.

### FIELD OF THE INVENTION

The present invention provides a novel formulation for reducing, ameliorating or treating a condition associated with ageing, such as chronic pulmonary obstructive disease (COPD), and methods of making and using the same.

### BACKGROUND OF THE INVENTION

Every mammal ages, and at present, researchers are only just beginning to understand the biological basis of ageing even in relatively simple and short-lived organisms such as yeast. Less still is known of mammalian ageing, in part due to the much longer lives of even small mammals such as the mouse (around 3 years). The factors proposed to influence biological ageing fall into two main categories, programmed and damage-related. Programmed factors follow a biological timetable, perhaps one that might be a continuation of the one that regulates childhood growth and development. This regulation would depend on changes in gene expression that affect the systems responsible for maintenance, repair and defense responses. Damage-related factors include internal and environmental assaults to living organisms that induce cumulative damage at various levels.

There are three main metabolic pathways which can influence the rate of ageing: the FOXO3/Sirtuin pathway, probably responsive to caloric restriction, the Growth hormone/Insulin-like growth factor 1 signaling pathway, and the activity levels of the electron transport chain in mitochondria and (in plants) in chloroplasts. It is likely that most of these pathways affect ageing separately (see, e.g., Guarente, Leonard P.; Partridge, Linda; Wallace, Douglas C. (2008), Molecular Biology of Aging, New York: Cold Spring Harbor, pp. 347-362).

A number of characteristic ageing symptoms are experienced by a majority or by a significant proportion of humans during their lifetimes, including Hearing loss; Cognitive decline; Presbyopia; Pattern hair loss by the age of 50 affects about half of males and a quarter of females; Osteoarthritis; Cataracts; Frailty; Atherosclerosis; and Dementia.

Chronic obstructive pulmonary disease (COPD) is another disease common among ageing population, which includes chronic bronchitis and emphysema and is a chronic lung disease that makes it hard to breathe. The disease is increasingly common, affecting millions of Americans, and is the third leading cause of death in the U.S. COPD is a major cause of disability. Currently, millions of people are diagnosed with COPD. Many more people may have the disease and not even know it.

COPD develops slowly. Symptoms of COPD often worsen over time and can limit one's ability to do routine activities. Severe COPD may prevent one from doing even basic activities like walking, cooking, or taking care of himself/herself.

COPD may be influenced by race, ethnicity, gender, and environmental factors as well as genetic factors. A small fraction of COPD is hereditary (genetic), and air pollution and smoking are the two main environmental causes of COPD. Given that a larger and larger portion of the population is becoming car-drivers, it is expected that COPD will further spread.

COPD symptoms often don't appear until significant lung damage has occurred, and they usually worsen over time, particularly if smoking exposure continues. For chronic bronchitis, the main symptom is a daily cough and mucus (sputum) production at least three months a year for two consecutive years.

In about 1 percent of people with COPD, the disease results from a genetic disorder that causes low levels of a protein called alpha-1-antitrypsin. Alpha-1-antitrypsin (AAt) is made in the liver and secreted into the bloodstream to help protect the lungs. Alpha-1-antitrypsin deficiency can affect the liver as well as the lungs. Damage to the lung can occur in infants and children, not only adults with long smoking histories.

For adults with COPD related to AAt deficiency, treatment options include those used for people with more-common types of COPD. In addition, some people can be treated by replacing the missing AAt protein, which may prevent further damage to the lungs.

COPD can cause many complications, including:
Respiratory infections. People with COPD are more likely to catch colds, the flu and pneumonia. Any respiratory infection can make it much more difficult to breathe and could cause further damage to lung tissue. An annual flu vaccination and regular vaccination against pneumococcal pneumonia can prevent some infections.
Heart problems. For reasons that aren't fully understood, COPD can increase one's risk of heart disease, including heart attack. Quitting smoking may reduce this risk.
Lung cancer. People with COPD have a higher risk of developing lung cancer. Quitting smoking may reduce this risk.
High blood pressure in lung arteries. COPD may cause high blood pressure in the arteries that bring blood to the lungs (pulmonary hypertension).
Depression. Difficulty breathing can keep one from doing activities that you enjoy. And dealing with serious illness can contribute to development of depression.

Therefore, there is a continuing need for compositions and methods to treat or ameliorate a condition associated with ageing.

The embodiments described below address the above identified issues and need.

### SUMMARY OF THE INVENTION

In one aspect of the present invention, it is provided a composition comprising nicotinamide adenine dinucleotide (NAD+) and optionally a nonsteroidal anti-inflammatory drug (NSAID), wherein the NAD+is in an amount effective for a condition in a mammal associated with aging.

In some embodiments of the invention composition, optionally in combination with any of the various embodiments disclosed herein, the composition comprises the NSAID, wherein the NSAID is in an anti-inflammatorily effective amount.

In some embodiments of the invention composition, optionally in combination with any of the various embodiments disclosed herein, the condition is COPD, wherein the composition is formulated as a formulation for pulmonary delivery, wherein the NAD+is in an effective amount to cause the bronchial tubes and alveoli fibers in a mammal to restore elasticity so as to treat or ameliorate the COPD.

In some embodiments of the invention composition, optionally in combination with any of the various embodiments disclosed herein, the composition further comprises a carrier for pulmonary delivery.

In some embodiments of the invention composition, optionally in combination with any of the various embodiments disclosed herein, the pulmonary delivery is by nebulizer.

In some embodiments of the invention composition, optionally in combination with any of the various embodiments disclosed herein, the pulmonary delivery is by nasal spray or nasal inhaling.

In some embodiments of the invention composition, optionally in combination with any of the various embodiments disclosed herein, the pulmonary delivery is by an inhaler.

In some embodiments of the invention composition, optionally in combination with any of the various embodiments disclosed herein, the composition is in a subcutaneous pellet form of injections for long term delivery.

In some embodiments of the invention composition, optionally in combination with any of the various embodiments disclosed herein, the COPD is chronic bronchitis.

In some embodiments of the invention composition, optionally in combination with any of the various embodiments disclosed herein, the COPD is emphysema.

In some embodiments of the invention composition, optionally in combination with any of the various embodiments disclosed herein, the condition is hair loss.

In some embodiments of the invention composition, optionally in combination with any of the various embodiments disclosed herein, the mammal is a human being.

In another aspect of the present invention, it is provided a method for treating or ameliorating a condition in a mammal associated with aging, said method comprising administering to said mammal a composition comprising nicotinamide adenine dinucleotide (NAD+) and optionally a nonsteroidal anti-inflammatory drug (NSAID), wherein the NAD+ is in an amount effective for the condition.

In some embodiments of the invention method, optionally in combination with any of the various embodiments disclosed herein, the composition comprises the NSAID, wherein the NSAID is in an anti-inflammatorily effective amount.

In some embodiments of the invention method, optionally in combination with any of the various embodiments disclosed herein, the condition is COPD, wherein the composition is formulated as a formulation for pulmonary delivery, wherein the NAD+ is in an effective amount to cause the bronchial tubes and alveoli fibers in a mammal to restore elasticity so as to treat or ameliorate the COPD.

In some embodiments of the invention method, optionally in combination with any of the various embodiments disclosed herein, the composition is in a subcutaneous pellet form of injections for long term delivery.

In some embodiments of the invention method, optionally in combination with any of the various embodiments disclosed herein, the composition comprises further comprises a carrier for pulmonary delivery.

In some embodiments of the invention method, optionally in combination with any of the various embodiments disclosed herein, the pulmonary delivery is by nebulizer.

In some embodiments of the invention method, optionally in combination with any of the various embodiments disclosed herein, the pulmonary delivery is by nasal spray or nasal inhaling.

In some embodiments of the invention method, optionally in combination with any of the various embodiments disclosed herein, the pulmonary delivery is by an inhaler.

In some embodiments of the invention method, optionally in combination with any of the various embodiments disclosed herein, the COPD is chronic bronchitis.

In some embodiments of the invention method, optionally in combination with any of the various embodiments disclosed herein, the COPD is emphysema.

In some embodiments of the invention method, optionally in combination with any of the various embodiments disclosed herein, the condition is hair loss.

In some embodiments of the invention method, optionally in combination with any of the various embodiments disclosed herein, the mammal is a human being.

In a further aspect of the present invention, it is provided a method of fabricating a composition, comprising providing nicotinamide adenine dinucleotide (NAD+) and forming a composition, wherein the composition comprises the nicotinamide adenine dinucleotide (NAD+) and optionally a nonsteroidal anti-inflammatory drug (NSAID), wherein the NAD+ is in an amount effective for a condition in a mammal associated with aging.

In some embodiments of the invention method, optionally in combination with any of the various embodiments disclosed herein, the composition comprises the NSAID, wherein the NSAID is in an anti-inflammatorily effective amount.

In some embodiments of the invention method, optionally in combination with any of the various embodiments disclosed herein, the condition is COPD, wherein the composition is formulated as a formulation for pulmonary delivery, wherein the NAD+ is in an effective amount to cause the bronchial tubes and alveoli fibers in a mammal to restore elasticity so as to treat or ameliorate the COPD.

In some embodiments of the invention method, optionally in combination with any of the various embodiments disclosed herein, the composition comprises further comprises a carrier for pulmonary delivery.

In some embodiments of the invention method, optionally in combination with any of the various embodiments disclosed herein, the pulmonary delivery is by nebulizer.

In some embodiments of the invention method, optionally in combination with any of the various embodiments disclosed herein, the pulmonary delivery is by nasal spray or nasal inhaling.

In some embodiments of the invention method, optionally in combination with any of the various embodiments disclosed herein, the pulmonary delivery is by an inhaler.

In some embodiments of the invention method, optionally in combination with any of the various embodiments disclosed herein, the composition is in a subcutaneous pellet form of injections for long term delivery.

In some embodiments of the invention method, optionally in combination with any of the various embodiments disclosed herein, the COPD is chronic bronchitis.

In some embodiments of the invention method, optionally in combination with any of the various embodiments disclosed herein, the COPD is emphysema.

In some embodiments of the invention method, optionally in combination with any of the various embodiments disclosed herein, the condition is hair loss.

In some embodiments of the invention method, optionally in combination with any of the various embodiments disclosed herein, the mammal is a human being.

### DETAILED DESCRIPTION OF THE INVENTION

### Definitions

As used herein, the terms “administering” or “administration” of an agent, drug, or peptide to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the term “rapid release” shall mean the release dosage forms for which ≥80% (e.g., about 80%, 85%, 90%, 95%, 98% or 99%) of labelled amount releases within 30 min. Conversely, the term “sustained release” shall mean the release dosage forms for which ≥80% (e.g., about 80%, 85%, 90%, 95%, 98% or 99%) of labelled amount releases over a period ≥30 minutes, e.g., about 45 minutes, 60 minutes, 90 minutes, 120 minutes, 2 hours, 4 hours, 6 hours, 8 hours, 12 hours, 24 hours, 2 days, 3 days, 4 days, 5 days, 6 days, 7 days, or beyond. The term “rapid release” is used interchangeably with the term “rapid delivery”, “fast release”, “fast delivery”, “immediate release”, or “immediate delivery.” The term “sustained release” is used interchangeably with the term “sustained delivery”, “long term release”, “long term delivery”, “delayed release”, or “delayed delivery.”

Pulmonary delivery of drug has become an attractive target as the lung is capable of absorbing pharmaceuticals either for local deposition or for systemic delivery. The pulmonary route as a non-invasive administration is suitable for systemic and local delivery of therapeutic agents, because the high permeability and large absorptive surface area of lungs, and good blood supply. As used herein, the term “pulmonary delivery” includes drug delivery via nasal spray, nasal inhaling, an oral inhaler, or any other drug delivery techniques capable of causing a drug to be delivered to the respiratory track and/or lung.

As used herein, the terms “disease,” “disorder,” or “complication” refers to any deviation from a normal state in a subject. In preferred embodiments, the methods and compositions of the present invention are useful in the diagnosis and treatment of diseases where ageing is involved. The present invention finds use with any number of diseases including, but not limited to, COPD.

As used herein, by the term “effective amount,” “amount effective,” “therapeutically effective amount,” or the like, it is meant an amount effective at dosages and for periods of time necessary to achieve the desired result. Generally, for NAD+, such effective amount refers to an amount of NAD+ that is capable of increasing blood level NAD+/NADH ratio to have a therapeutically significant effect on a liver disorder in a mammal subject. Examples of such effective amount range from about 1 μg to about 10 grams (e.g., about 2 μg, 5 μg, 10 μg, 20 μg, 50 μg, 100 μg, 200 μg, 500 μg, 1 mg, 10 mg, 20 mg, 50 mg, 100 mg, 200 mg, 500 mg, or about 1000 mg) per kilogram body weight.

As used herein, the term “anti-inflammatorily effective” shall mean effective for reducing, ameliorating, or treating inflammation. In this context, the term “anti-inflammatorily amount” shall mean an amount effective at dosages and for periods of time necessary to achieve anti-inflammation. Note, while an NSAID can be used for indications other than inflammation, e.g., pain or fever, the effective amount of the NSAID for pain or fever is different from the effective amount for anti-inflammation as anti-inflammation amount of NSAID is generally higher than that for pain, fever, and in the case of Aspirin, blood-thinning. Generally, in combination with NAD+ in a composition of invention, an “anti-inflammatorily effective” of NSAID falls within the range of 10 μg to about 10 grams (e.g., about 20 μg, 50 μg, 100 μg, 200 μg, 500 μg, 1 mg, 10 mg, 20 mg, 50 mg, 100 mg, 200 mg, 500 mg, 1000 mg, 2000 mg, or 5000 mg) per kilogram body weight.

As used herein, a “formulation,” “pharmaceutical formulation” all include a composition comprising at least one of NAD+ or a precursor thereof. Optionally, the “composition,” “pharmaceutical composition” or “therapeutic agent” further comprises pharmaceutically acceptable diluents or carriers.

As used herein, the term “preventing” means causing the clinical symptoms of the disease state not to develop, e.g., inhibiting the onset of disease, in a subject that may be exposed to or predisposed to the disease state, but does not yet experience or display symptoms of the disease state.

As used herein, the term “subject” refers to any animal (e.g., a mammal), including, but not limited to, humans, non-human primates, rodents, and the like, which is to be the recipient of a particular treatment.

As used herein, the terms “treating” or “treatment” or “alleviation” refers to both therapeutic treatment and prophylactic or preventative measures, wherein the object is to prevent or slow down (lessen) the targeted pathologic condition or disorder.

As used herein, the terms “composition” and “formulation” are sometimes used interchangeably.

### Causes of COPD

The main cause of COPD in developed countries is tobacco smoking. In the developing world, COPD often occurs in people exposed to fumes from burning fuel for cooking and heating in poorly ventilated homes.

Only about 20 to 30 percent of chronic smokers may develop clinically apparent COPD, although many smokers with long smoking histories may develop reduced lung function. Some smokers develop less common lung conditions. They may be misdiagnosed as having COPD until a more thorough evaluation is performed.

The below describes how the lungs are affected by COPD.

Air travels down the windpipe (trachea) and into the lungs through two large tubes (bronchi). Inside the lungs, these tubes divide many times—like the branches of a tree—into many smaller tubes (bronchioles) that end in clusters of tiny air sacs (alveoli).

The air sacs have very thin walls full of tiny blood vessels (capillaries). The oxygen in the air one inhales passes into these blood vessels and enters the bloodstream. At the same time, carbon dioxide—a gas that is a waste product of metabolism—is exhaled.

The lungs rely on the natural elasticity of the bronchial tubes and air sacs to force air out of the body. COPD causes them to lose their elasticity and overexpand, which leaves some air trapped in the lungs when one exhales.

### Nicotinamide Adenine Dinucleotide

Nicotinamide adenine dinucleotide (NAD) is a coenzyme found in all living cells. The compound is a dinucleotide, because it consists of two nucleotides joined through their phosphate groups. One nucleotide contains an adenine base and the other nicotinamide. Nicotinamide adenine dinucleotide exists in two forms, an oxidized and reduced form abbreviated as NAD+ and NADH respectively, as shown below:

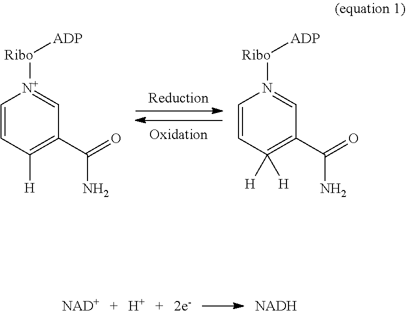

In metabolism, nicotinamide adenine dinucleotide is involved in redox reactions, carrying electrons from one reaction to another. The coenzyme is, therefore, found in two forms in cells: NAD+ is an oxidizing agent—it accepts electrons from other molecules and becomes reduced. This reaction forms NADH, which can then be used as a reducing agent to donate electrons. These electron-transfer reactions are the main function of NAD. However, it is also used in other cellular processes, the most notable one being a substrate of enzymes that add or remove chemical groups from proteins, in posttranslational modifications. Because of the importance of these functions, the enzymes involved in NAD metabolism are targets for drug discovery.

In organisms, NAD can be synthesized from simple building-blocks (de novo) from the amino acids tryptophan or aspartic acid. In an alternative route, more complex components of the coenzymes are taken up from food as vitamin niacin. Similar compounds are released by reactions that break down the structure of NAD. These preformed components then pass through a salvage pathway that recycles them back into the active form. Some NAD is also converted into nicotinamide adenine dinucleotide phosphate (NADP); the chemistry of this related coenzyme is similar to that of NAD, but it has different roles in metabolism.

Although NAD+ is written with a superscript plus sign because of the formal charge on a particular nitrogen atom, at physiological pH for the most part it is actually a singly charged anion (charge of minus 1), while NADH is a doubly charged anion.

### Regulation of SIRT1 by NAD+

While the functions of NAD+ are the focus of many studies, it is believed the mechanism of action of NAD+ with respect to the invention disclosed herein is its ability to modulate the level of SIRT1. A decrease of SIRT1 level, in turn, reportedly is linked to many disorders including aging-associated disorders (see, e.g., Ng, F., et al., SIRT1 in the brain—connections with aging-associated disorders and lifespan, in Frontiers in Cellular Neuroscience, March 2015, vol. 9, Article 64 (Review). NAD has been reported to reverse aging in that it enables communication inside cells between the nucleus and mitochondria (https://hms.harvard.edu/news/genetics/new-reversible-cause-aging-12-19-13). A study reported in 2012 there exists a link between oxidative stress and PARP activity, aging, and a decline in NAD+ levels in human tissue (Massudi, H., et al., PLoS One, July 2012, Vol. 7 (7): (e42357). NAD's ability to restore cell communication to reverse the aging process is also confirmed in another report (https://www.theguardian.com/science/2013/dec/20/anti-ageing-human-trials).

While not intending to be bound by a specific theory, Applicant found that NAD is effective to restore elasticity of the bronchial tubes and air sacs of COPD patients. As such, administration of NAD+ to a subject having COPD is effective to treat or ameliorate COPD. This is consistent with the studies that NAD+ is anti-aging.

As used herein, while the term “NAD” is sometimes used instead of NAD+, the reference to NAD in the instant application shall mean the net amount of NAD+ in the NAD is higher than NADH in that the molar ratio of NAD+/NADH in the NAD >1.

### Nonsteroidal Anti-inflammatory Drugs (NSAIDs)

In an aspect of the present invention, NAD+can be used together with a NSAID. Synergistically, NAD+ regulates SIRT1, which is key in the aging process, the NSAID inhibits or treats inflammation, which is causally related to aging (see, Jenny, N S, Discov Med. 2012 June; 13(73):451-60) (Review). As such, the invention composition is not only effective to regulate SIRT1, it is also anti-inflammatorily effective, resulting in enhanced anti-aging effect.

Nonsteroidal anti-inflammatory drugs (NSAIDs) are a drug class that groups together drugs that provide analgesic (pain-killing) and antipyretic (fever-reducing) effects, and, in higher doses, anti-inflammatory effects. The most prominent members of this group of drugs are aspirin, ibuprofen and naproxen, all available over the counter in most countries (Buer, J. K. (October 2014), Inflammopharmacology, 22 (5): 263-7). Most NSAIDs inhibit the activity of cyclooxygenase-1 (COX-1) and cyclooxygenase-2 (COX-2), and thereby the synthesis of prostaglandins and thromboxanes. It is thought that inhibiting COX-2 leads to the anti-inflammatory, analgesic and antipyretic effects and that those NSAIDs also inhibiting COX-1, particularly aspirin, may cause gastrointestinal bleeding and ulcers.

Celecoxib (Celebrex) belongs to a newer class of NSAIDs, which doctors call a “COX-2 inhibitor” or a “COX-2 selective” NSAID.

### Pharmaceutical Compositions/Formulation

In one aspect of the present invention, it is provided a composition comprising nicotinamide adenine dinucleotide (NAD+) and optionally a nonsteroidal anti-inflammatory drug (NSAID), wherein the NAD+ is in an amount effective for a condition in a mammal associated with aging.

In some embodiments of the invention composition, optionally in combination with any of the various embodiments disclosed herein, the composition comprises the NSAID, wherein the NSAID is in an anti-inflammatorily effective amount.

In some embodiments of the invention composition, optionally in combination with any of the various embodiments disclosed herein, the condition is COPD, wherein the composition is formulated as a formulation for pulmonary delivery, wherein the NAD+ is in an effective amount to cause the bronchial tubes and alveoli fibers in a mammal to restore elasticity so as to treat or ameliorate the COPD.

In some embodiments of the invention composition, optionally in combination with any of the various embodiments disclosed herein, the composition further comprises a carrier for pulmonary delivery.

In some embodiments of the invention composition, optionally in combination with any of the various embodiments disclosed herein, the pulmonary delivery is by nebulizer.

In some embodiments of the invention composition, optionally in combination with any of the various embodiments disclosed herein, the pulmonary delivery is by nasal spray or nasal inhaling.

In some embodiments of the invention composition, optionally in combination with any of the various embodiments disclosed herein, the pulmonary delivery is by an inhaler.

In some embodiments of the invention composition, optionally in combination with any of the various embodiments disclosed herein, the composition is in a subcutaneous pellet form of injections for long term delivery.

In some embodiments of the invention composition, optionally in combination with any of the various embodiments disclosed herein, the COPD is chronic bronchitis.

In some embodiments of the invention composition, optionally in combination with any of the various embodiments disclosed herein, the COPD is emphysema.

In some embodiments of the invention composition, optionally in combination with any of the various embodiments disclosed herein, the condition is hair loss.

In some embodiments of the invention composition, optionally in combination with any of the various embodiments disclosed herein, the mammal is a human being.

The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these compositions can contain suitable pharmaceutically acceptable carriers comprising excipients and auxiliaries which facilitate processing of the active compounds into preparations which can be used pharmaceutically. Compositions of the invention can be administered by any number of routes including, but not limited to, oral, inhaling (e.g., pulmonary or nasal), injection, intravenous, intramuscular, intra-arterial, intramedullary, intrathecal, intraventricular, transdermal, subcutaneous, intraperitoneal, intranasal, parenteral, topical, sublingual, or rectal means.

The composition of invention can be formulated for rapid release or sustained release (AKA long term delivery) formulations. Compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. An example of injection formulation is subcutaneous pellet form of injections for long term delivery.

Further details on techniques for formulation and administration can be found in the latest edition of REMINGTON'S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

It is noted that the compositions and methods disclosed herein may be administered to any subject as defined herein. In one embodiment, the subject is human. In another embodiment, the subject is a pet, e.g., cat, dog, or the like, and in a particular embodiment, is an overweight pet or animal. It is further noted that a corresponding composition, e.g., a pharmaceutical composition, may be provided for use in any method described herein.

All forms of NAD coenzyme are white amorphous powders that are hygroscopic and highly water-soluble. The solids are stable if stored dry and in the dark. Solutions of NAD+ are colorless and stable for about a week at 4° C. and neutral pH, but decompose rapidly in acids or alkalis. Upon decomposition, they form products that are enzyme inhibitors. Various formulations therefore can be made making use of such properties of NAD.

Those skilled in the art will appreciate that numerous delivery mechanisms are available for delivering a therapeutic agent to an area of need. By way of example, the agent may be delivered using a liposome as the delivery vehicle. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human.

### Method of Fabrication

In a further aspect of the present invention, it is provided a method of fabricating a composition, comprising providing nicotinamide adenine dinucleotide (NAD+) and forming a composition, wherein the composition comprises the nicotinamide adenine dinucleotide (NAD+) and optionally a nonsteroidal anti-inflammatory drug (NSAID), wherein the NAD+ is in an amount effective for a condition in a mammal associated with aging.

In some embodiments of the invention method, optionally in combination with any of the various embodiments disclosed herein, the composition comprises the NSAID, wherein the NSAID is in an anti-inflammatorily effective amount.

In some embodiments of the invention method, optionally in combination with any of the various embodiments disclosed herein, the condition is COPD, wherein the composition is formulated as a formulation for pulmonary delivery, wherein the NAD+ is in an effective amount to cause the bronchial tubes and alveoli fibers in a mammal to restore elasticity so as to treat or ameliorate the COPD.

In some embodiments of the invention method, optionally in combination with any of the various embodiments disclosed herein, the composition comprises further comprises a carrier for pulmonary delivery.

In some embodiments of the invention method, optionally in combination with any of the various embodiments disclosed herein, the pulmonary delivery is by nebulizer.

In some embodiments of the invention method, optionally in combination with any of the various embodiments disclosed herein, the pulmonary delivery is by nasal spray or nasal inhaling.

In some embodiments of the invention method, optionally in combination with any of the various embodiments disclosed herein, the pulmonary delivery is by an inhaler.

In some embodiments of the invention method, optionally in combination with any of the various embodiments disclosed herein, the composition is in a subcutaneous pellet form of injections for long term delivery.

In some embodiments of the invention method, optionally in combination with any of the various embodiments disclosed herein, the COPD is chronic bronchitis.

In some embodiments of the invention method, optionally in combination with any of the various embodiments disclosed herein, the COPD is emphysema.

In some embodiments of the invention method, optionally in combination with any of the various embodiments disclosed herein, the condition is hair loss.

In some embodiments of the invention method, optionally in combination with any of the various embodiments disclosed herein, the mammal is a human being.

### Method of Use

In another aspect of the present invention, it is provided a method for treating or ameliorating a condition in a mammal associated with aging, said method comprising administering to said mammal a composition comprising nicotinamide adenine dinucleotide (NAD+) and optionally a nonsteroidal anti-inflammatory drug (NSAID), wherein the NAD+ is in an amount effective for the condition.

In some embodiments of the invention method, optionally in combination with any of the various embodiments disclosed herein, the composition comprises the NSAID, wherein the NSAID is in an anti-inflammatorily effective amount.

In some embodiments of the invention method, optionally in combination with any of the various embodiments disclosed herein, the condition is COPD, wherein the composition is formulated as a formulation for pulmonary delivery, wherein the NAD+ is in an effective amount to cause the bronchial tubes and alveoli fibers in a mammal to restore elasticity so as to treat or ameliorate the COPD.

In some embodiments of the invention method, optionally in combination with any of the various embodiments disclosed herein, the composition is in a subcutaneous pellet form of injections for long term delivery.

In some embodiments of the invention method, optionally in combination with any of the various embodiments disclosed herein, the composition comprises further comprises a carrier for pulmonary delivery.

In some embodiments of the invention method, optionally in combination with any of the various embodiments disclosed herein, the pulmonary delivery is by nebulizer.

In some embodiments of the invention method, optionally in combination with any of the various embodiments disclosed herein, the pulmonary delivery is by nasal spray or nasal inhaling.

In some embodiments of the invention method, optionally in combination with any of the various embodiments disclosed herein, the pulmonary delivery is by an inhaler.

In some embodiments of the invention method, optionally in combination with any of the various embodiments disclosed herein, the COPD is chronic bronchitis.

In some embodiments of the invention method, optionally in combination with any of the various embodiments disclosed herein, the COPD is emphysema.

In some embodiments of the invention method, optionally in combination with any of the various embodiments disclosed herein, the condition is hair loss.

In some embodiments of the invention method, optionally in combination with any of the various embodiments disclosed herein, the mammal is a human being.

### Disorders

The composition disclosed herein can be used to treat or ameliorate any disorder or conditions associated with ageing, in particular, COPD. Various types of COPD are described above.

### Kit/Dosage Form

In a further aspect of the present invention, it is provided a kit, comprising:
a formulation comprising a carrier and an effective amount of nicotinamide adenine dinucleotide (NAD) via pulmonary delivery to cause the bronchial tubes and alveoli fibers to restore elasticity so as to treat or ameliorate the COPD,
a dosing unit that provides one or more doses of the formulation, each dose providing the effective amount of nicotinamide adenine dinucleotide (NAD) via pulmonary delivery to cause the bronchial tubes and alveoli fibers to restore elasticity so as to treat or ameliorate the COPD,
an optional liquid housing unit for housing an optional liquid carrier, and
a dispensing unit for dispensing the formulation in the dosing unit for pulmonary application to a subject in need thereof,
wherein the dosage unit, the optional liquid housing unit, and the dispensing unit are separate, partially joined, or entirely joined forming a single structure.

In some embodiments of the invention kit, optionally in combination with any or all of the various embodiments disclosed herein, the application is nasal administration.

In some embodiments of the invention formulation, optionally in combination with any or all of the various embodiments disclosed herein, the liquid carrier is water.

In some embodiments of the invention kit, optionally in combination with any or all of the various embodiments disclosed herein, the pulmonary delivery is by nebulizer.

In some embodiments of the invention kit, optionally in combination with any or all of the various embodiments disclosed herein, the pulmonary delivery is by nasal spray or nasal inhaling.

In some embodiments of the invention kit, optionally in combination with any or all of the various embodiments disclosed herein, the COPD is chronic bronchitis.

In some embodiments of the invention kit, optionally in combination with any or all of the various embodiments disclosed herein, the COPD is emphysema.

In some embodiments of the invention kit, optionally in combination with any or all of the various embodiments disclosed herein, the mammal is a human being.

The following examples illustrate rather than limit the embodiments of the present invention.

### EXAMPLE

### Example 1

### Studies of the Invention Composition for COPD

A male subject, who is 55 years old and suffers from COPD since childhood due to second-hand smoking, was administered twice a day by nasal spray a composition of invention (with a dosage of 50 mg NAD+ per administration) continuously for a week. Significant improvement of symptoms of COPD after the 3-day time point. After a week (7 days), morning coughing, the major part of the symptoms of COPD that the patient suffers, stopped entirely.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The teachings of the references, including patents and patent related documents, cited herein are incorporated herein in their entirety to the extent not inconsistent with the teachings herein.

I claim:

1. A composition comprising nicotinamide adenine dinucleotide (NAD+) and optionally a nonsteroidal anti-inflammatory drug (NSAID), wherein the NAD+ is in an amount effective for a condition in a mammal associated with aging.

2. The composition according to claim 1, comprising the NSAID, wherein the NSAID is in an anti-inflammatorily effective amount.

3. The composition of claim 1, wherein the condition is COPD, wherein the composition is formulated as a formulation for pulmonary delivery, wherein the NAD+ is in an effective amount to cause the bronchial tubes and alveoli fibers in a mammal to restore elasticity so as to treat or ameliorate the COPD.

4. The composition of claim 3, further comprising a carrier for pulmonary delivery.

5. The composition of claim 1, wherein the pulmonary delivery is by nebulizer.

6. The composition of claim 1, wherein the pulmonary delivery is by nasal spray or nasal inhaling.

7. The composition of claim 1, wherein the pulmonary delivery is by an inhaler.

8. The composition of claim 3, wherein the COPD is chronic bronchitis.

9. The composition of claim 3, wherein the COPD is emphysema.

10. The composition of claim 2, wherein the condition is hair loss.

11. The composition of claim 1, which is in a subcutaneous pellet form of injections for long term delivery.

12. A method for treating or ameliorating a condition in a mammal associated with aging, said method comprising administering to said mammal a composition comprising nicotinamide adenine dinucleotide (NAD+) and optionally a nonsteroidal anti-inflammatory drug (NSAID), wherein the NAD+ is in an amount effective for the condition.

13. The method of claim 12, wherein the composition comprises the NSAID, wherein the NSAID is in an anti-inflammatorily effective amount.

14. The method of claim 12, wherein the condition is COPD, wherein the composition is formulated as a formulation for pulmonary delivery, wherein the NAD+ is in an effective amount to cause the bronchial tubes and alveoli fibers in a mammal to restore elasticity so as to treat or ameliorate the COPD.

15. The method of claim 13, wherein the composition comprises further comprises a carrier for pulmonary delivery.

16. The method of claim 15, wherein the pulmonary delivery is by nebulizer.

17. The method of claim 15, wherein the pulmonary delivery is by nasal spray or nasal inhaling.

18. The method of claim 15, wherein the pulmonary delivery is by an inhaler.

19. The method of claim 14, wherein the COPD is chronic bronchitis.

20. The method of claim 14, wherein the COPD is emphysema.

21. The method of claim 13, wherein the condition is hair loss.

22. The method of claim 12, wherein the composition is in a subcutaneous pellet form of injections for long term delivery.

23. A method of fabricating a composition, comprising providing nicotinamide adenine dinucleotide (NAD+) and forming a composition according to claim 1.
